(19) United States
(12) Patent Application Publication
(10) Pub. No.: US 2012/0101460 A1
(43) Pub. Date: Apr. 26, 2012

(54) FLEXIBLE RESILIENT ABSORBENT CELLULOSIC NONWOVEN STRUCTURE

(75) Inventors: Ralf Ehmke, Meyenburg (DE); Henning Röttger, Kaltenkirchen (DE)

(73) Assignee: Glatfelter Falkenhagen GmbH, Pritzwalk (DE)

(21) Appl. No.: 13/381,688

(22) PCT Filed: Jul. 1, 2010
(86) PCT No.: PCT/EP2010/059361

§ 371 Date: Dec. 30, 2011

Related U.S. Application Data
(60) Provisional application No. 61/222,278, filed on Jul. 1, 2009.

(51) Int. Cl. A61L 15/28; B27N 3/04

(52) U.S. Cl. 604/374; 156/62.2

(57) ABSTRACT

The invention relates to a fibrous porous fluid absorbent material comprising a nonwoven, in particular made by an Airlaid process and comprising fibers at least 50% of said fibers being cellulosic fibers. The fibrous porous fluid absorbent material comprises a core wherein said fibers of said core are non-connected or only partially connected and crosslinked with each other and at least one perforated surface layer having a perforation. The fibers of the surface layer are connected to each other and the flexural rigidity of said fibrous porous fluid absorbent material measured according to EDANA Standard Test Method 90.5 is reduced by at least 20%, preferably by at least 30%, more preferably by at least 40%, most preferably by at least 50%, compared to a corresponding non perforated fibrous porous fluid absorbent material comprising a core and a non perforated surface layer.

### FIELD OF THE INVENTION

The present invention relates to a fluid-absorbent multi-layered nonwoven fabric structure in particular made of cellulosic fibers, manufactured with an Airlaid process. Such nonwoven fabric structure hereinafter is simply called “nonwoven”, “Airlaid material” or “Airlaid structure”. Airlaid thus designates the process as well as the product.

The nonwoven according to the present invention can comprise further powders, particles or fibrous materials in order to give that structure desired specific properties. Super absorbent polymers are in this context of particular interest to enhance fluid absorption capacity of the structure even under load.

### BACKGROUND

Cellulosic nonwoven structures of this type are used in personal care and feminine hygiene products for single use, such as panty liners, sanitary napkins, incontinence articles or diapers as fluid absorbent core. Of high interest especially for this application area is the possibility to integrate superabsorbent polymers into this absorbent cellulosic nonwoven structure.

The present invention is directed to creating a fluid-absorbent structure having a very good resiliency not only perpendicular to the surface but also in parallel to the surface providing a certain resistance against getting crumpled. The material according to the invention has a restoring force that brings the material back in its original flat form after crumpling. This feature is particularly advantageous for the wear comfort of the hygiene articles, because there is a significantly improved body-fit of the sanitary article. If the hygiene products are in use, they are constantly in movement and the article has to adapt shape to the body of the wearer.

The present invention is further directed to create a fluid-absorbent structure having a very good flexibility which can be adjusted in specific zones. Adjusting the flexibility of an absorbent core in specific zones allows to predetermine how the core and the hygiene article (e.g. sanitary napkin, incontinence product, diaper) is going to change shape when exposed to a force generated by movement of the body of the wearer. This specific adjustment of the flexibility of the inventive material can be achieved by changing the structure of the surface e.g. by means of perforation.

State of the art hygiene products typically use one of the following materials as fluid absorbent structures:
Individualized cellulosic fibers generated by mechanical opening of wood pulp obtained by means of hammer mills, being deposited as stripes in hygiene products (fluff-pulp). Super absorbent powder (SAP) or super absorbent fibers (SAF) are added to this fluff-pulp to increase the liquid absorption capacity, particularly under pressure. The fluff pulp-based absorbent structures are bulky and have especially in the wet condition no mechanical integrity and can not recover to the original shape after mechanical deformation. This implies limited comfort in particular in products for incontinence and feminine hygiene, since the products are easily shaped to a bulky bundle especially after exposure to liquid (menses or urine) which uncomfortable and annoying especially when wearing closely fitting clothes.
Airlaid materials, which are essentially made from fluff wood pulp fibers. The Airlaid material can comprise superabsorbent materials (SAP or SAF) to increase the absorption capacity together with the fluff pulp. Compared to the formerly mentioned fluff-pulp-based products, hygiene products using Airlaid material as absorbent material are much thinner and therefore offer the wearer an increased comfort. In addition, especially Airlaid materials bonded with a chemical binder, thermoplastic binder fibers or both maintain their mechanical integrity even after exposure to liquids. Other thermoplastic materials like fibers and powder can be added to the Airlaid material to achieve additional functionality like odour and enhanced liquid absorption, compatibility to ultrasonic welding etc. However, these materials are quite stiff especially for products with a higher absorption capacity requiring thick materials with a high load of super absorbing polymers. To further improve the wear comfort of such hygiene products, it is desired to increase the softness, compression resistance, resiliency, drape and a certain resistance against crumpling.
To improve the softness of the absorbent structures of hygiene products open foam structures have been developed which offer a compression resistance and an enhanced resistance against crumpling.
U.S. Pat. No. 5,869,171 The Procter & Gamble Company, Shiveley, DesMarais, Dyer & Stone, Foam Heterogeneous Materials, filed 1998, May 29, discloses an absorbent article, in which high internal phase emulsions (HIPE) foams are used. HIPE is an emulsion, having an internal water phase as a discontinuous dispersed phase with volume fractions of more than 70% compared to the oil phase as an external phase. After polymerization crosslinked porous polymers can arise of these emulsions.

The liquid absorption of these foam structures results from capillary forces. Super absorbent polymers can be added for enhanced liquid absorption by processing of super absorbent polymers producing the foam or deposition of superabsorbent particles into the foam material; see EP1156837 B1 to Dow Global Technologies Inc., Gartner et al., “Manufacture of Superabsorbents in high internal phase emulsions”. Compared to pulp-based Airlaid materials, such foam structures are, especially if they have superabsorbtion capability, significantly more expensive and are not manufactured from environment-friendly cellulose-based materials.

### DESCRIPTION

Comparing the compression resistance of fibrous two-dimensional structures (e.g. Airlaid material) with the compression resistance of foam, one will observe a considerably higher specific restoring force for foams. This is because of a continuous polymer-network in the foam, causing an intermittent compression not only of the structure under the point of applied pressure but also a deformation of the neighboring areas which are stretched. The restoring force is the sum of the retractive force of the compressed area and the withdrawal force of the deformed environment.

Another aspect of fibrous two-dimensional structures is their stiffness (flexural rigidity) or resiliency, respectively.

The following methods of mechanical treatment are known to reduce the stiffness of fibrous structures. These methods however have disadvantages especially when applied to the described structure:
Embossing: The local compaction of the material forms embossing points or -lines, forming bending points or lines in the material reducing the overall stiffness of the material. This technology can be used in zones to achieve a specific deformation of the structure, e.g. in hygiene products. This technology has the disadvantage of local compaction of the material in the embossed zones destroying the desired softness and compression recovery of the material as well as significantly changing the fluid dynamics of the fibrous structure. This substantially reduces the benefits of the described structure.
Applying mechanical treatment, such as kneading, pressing and pulling or repeated bending of the material (e.g. by guiding the material along rods), the stiffness of materials can be reduced, too. These treatments will break connecting points in the structure of the material, which contribute to the mechanical stiffness and elastic bonding points will be activated. If this technology is applied to the described material, bonding points connecting the fibers which surface will be destroyed too, weakening the restoring force against a local deformation. Furthermore, this procedure in contrast to the embossing cannot be used locally, so that a specific adaptation of the article to the body shape is not possible.
There are other procedures which locally stretch the nonwoven or textile materials changing the textile properties and especially reducing the stiffness.
   U.S. Pat. No. 5,518,801 The Procter & Gamble Company, Chappell et al., Web material exhibiting elastic like behavior, discloses treatment of materials such as polyethylene film (cl.10) with the example FIG. 36, 37 rolls.
   WO2008050311 The Procter and Gamble Company, Hupp et al. Clothlike non-woven fibrous structures and processes for making same, for example, describes the treatment of non-woven materials similar to the above-mentioned film, in order to create a textile-like material. Mentioned processes include printing, embossing, laminating, slitting, perforating, cutting edges, stacking, folding, and mechanical softening.

According to these two technologies described in abovementioned patents, the material is guided through e.g. interlocking wheels, which have an additional surface structure. This treatment generates a local stretching of the material. This procedure is in principle also applicable to Airlaid materials but offers the disadvantage that the three-dimensional structure of the Airlaid is modified. Important properties for use in hygiene articles such as liquid distribution by capillary forces, also called wicking, changes undesirably. Especially in the use of Airlaid as absorbent component in hygiene products the pore structure is carefully designed and is important for the liquid distribution in the absorbent core.

It is an object of the invention to provide a fibrous porous fluid absorbent material, e.g. an Airlaid material that provides an increased restoring force while maintaining a certain resiliency.

According to the invention, this object is achieved by a fibrous porous fluid absorbent material comprising a nonwoven, in particular made by an Airlaid process and comprising fibers at least 50% of said fibers being cellulosic fibers, said fibrous porous fluid absorbent material comprising a core wherein said fibers of said core are non-connected or only partially connected and crosslinked with each other and at least one perforated surface layer having a perforation, wherein said fibers of said surface layer are connected to each other and wherein the flexural rigidity of said fibrous porous fluid absorbent material measured according to EDANA Standard Test Method 90.5 is reduced by at least 20%, preferably by at least 30%, more preferably by at least 40%, most preferably by at least 50%, compared to a corresponding non perforated fibrous porous fluid absorbent material comprising a core and a non perforated surface layer.

The invention includes the observation that in fibrous Airlaid structures, the individual cellulose fibers are not elastic by themselves and are only partially connected and crosslinked with each other e.g. by the addition of a liquid binder, the addition of binder fibers or binder particles.

If a punctual pressure is applied to such an Airlaid structure, the fibers below the pressure point will be compressed, but the fibers around the pressure point will at least partially re-orient responding to the applied pressure. These reoriented fibers only have a limited contribution to the retractive force of the compressed material so that total resorting force is limited and the material can be permanently deformed.

Surprisingly, it turns out that the restoring force against a local deformation of a structure such as fibrous Airlaid significantly increases when the fibers on both surfaces are connected with an additional preferred partially elastic surface. This surface can be formed by applying a binder partially penetrating the surface of the fibrous structure creating a network of connected fibers. Another suitable surface is achieved by adding a wetlaid tissue paper sprayed with binder to the surface of the fibrous material.

Definition: According to German standard DIN 6730 a wetlaid tissue paper is a product which in total or in part consists of cellulose fibers, with a fine, soft crepe and a closed formation creped out in paper machine with a dry content of more than 90%, made of one or more layers, very absorbent, having an area related mass of each layer of less than 25 g/m² before creping and having a wet crepe elongation of more than 5%.

ISO 12625-1 establishes general principles for the use of terms in the entire working field of tissue paper and tissue products. Here a common terminology in industry and commerce can be found.

According to INDA, the Association of The Nonwoven Fabrics Industry, the Airlaid or Air laid process is a nonwoven web forming process that disperses fibers into a fast moving air stream and condenses them onto a moving screen by means of pressure or vacuum. Further bonding steps consolidate this web into a stable nonwoven fabric also called Airlaid.

Wetlaid or wet laid process is a papermaking process made with a Fourdrinier Machine; transforming a source of wood pulp into a final paper product with the help of water.

Tissue paper is formed on such a paper machine having one single large steam heated drying cylinder called Yankee dryer.

Herein, the terms “wetlaid tissue paper”, “tissue paper”, “tissue product” and “tissue” are used as synonyms.

A product that comprises more than two interconnected layers of different materials is referred to as laminate.

According to a preferred embodiment of the invention the fibrous porous fluid absorbent material is a laminate having an asymmetric structure by means of lamination of a nonwoven material with an surface layer as carrier material being one component of the laminate, which can be chosen from the group of wetlaid tissue paper, or another nonwoven (e.g. melt blown, spun bond or combination thereof, carded, spun laced, i.e. hydro entangled etc.)

The surface layer or carrier material as one component of the laminate can be chosen from the group of wetlaid tissue paper, but also can be any other nonwoven (e.g. melt blown, spun bond or combination thereof, carded, spun laced, i.e. hydro entangled etc.). The surface layer can also be generated by application of a binder without adding additional material by connecting the fibers at the surface to a continuous network.

The increased force against compression, i.e. enhanced resiliency of the material after adding the surface layers is created by connecting the fibers in the neighborhood of the pressure point via the common surface to the deformed zone. The fibers connected to the surface can not respond to the deformation by reorientation. Therefore the fibers in the neighborhood of the pressure point contribute to the restoring force of the compressed fibers under the pressure point. If an elastic binder or surface layer is used to create the additional surface also the elongation of this elastic surface creates an additional restoring force as shown in FIG. 2.

As shown in FIG. 2 the laminate structure provides an increased resiliency of the material compared with the pure fiber structure. Unfortunately the formation of a laminate results in an increased flexural rigidity/stiffness. If the described structures are used in hygiene articles the increased stiffness is undesirable as it has a negative impact on the comfort (body fit) of the product.

The Airlaid structure of this invention has surprisingly shown that the flexibility of the structure can be significantly increased by perforating one or both surfaces in zones or across the full surface avoiding changes of the structure of the material between the two surfaces by avoiding too high compression of the material and reorientation of fibers.

According to a preferred embodiment of the invention a soft or elastic chemical binder dispersion is applied in order to provide integrity within the laminate components (e.g. wetlaid tissue paper combined with Airlaid). The absence of the binder dispersion could lead into weak integrity and further to a risk of sliding of the certain layers i.e. when pressure is applied.

Using an Airlaid material with one surface treated with a chemical binder (e.g. a binder dispersion) and a tissue layer treated with an elastic binder attached to the other surface it has been surprisingly revealed that the flexibility of the material is enhanced most if the tissue layer is perforated.

With respect to the perforation of the perforated surface layer, the following embodiments are preferred:
The fibrous porous fluid absorbent material has perforation holes, said perforation holes having a diameter that is smaller than 3 mm, preferably smaller than 1 mm, more preferably smaller than 0.5 mm, most preferably 0.2 mm.
The fibrous porous fluid absorbent material has perforation holes, said perforation holes being arranged with a distance between said perforation holes that is less than 30 mm and more than 0.5 mm, preferably smaller than 15 mm, more preferably smaller than 5 mm, most preferably 1-2 mm in diameter.
The fibrous porous fluid absorbent material has a thickness of less than 10 mm and more than 0.5 mm, preferably less than 5 mm, more preferably less than 3 mm, most preferably between 1 mm and 2 mm.
The fibrous porous fluid absorbent material perforation holes, wherein the perforation holes are produced by means of a perforation roller, e.g. a roller with needles.
The fibrous porous fluid absorbent material wherein the perforation of the surface layer is achieved by slits instead of punched holes.
The fibrous porous fluid absorbent material wherein the perforation of the surface layer is only provided in distinct zones of said fibrous porous fluid absorbent material.

### BRIEF DESCRIPTION OF THE DRAWINGS

FIG. 1 is an illustration of the behavior of an Airlaid material without a surface layer comprising interconnected fibers exposed to local pressure.

FIG. 2 is an illustration of the behavior of an Airlaid material with a surface layer comprising interconnected fibers exposed to local pressure.

FIG. 3 is an illustration of the behaviour of an Airlaid material with a perforated surface layer comprising interconnected fibers exposed to local pressure.

FIG. 4 is an exemplary top view of a perforated surface layer according to the invention.

### DETAILED DESCRIPTION OF THE INVENTION

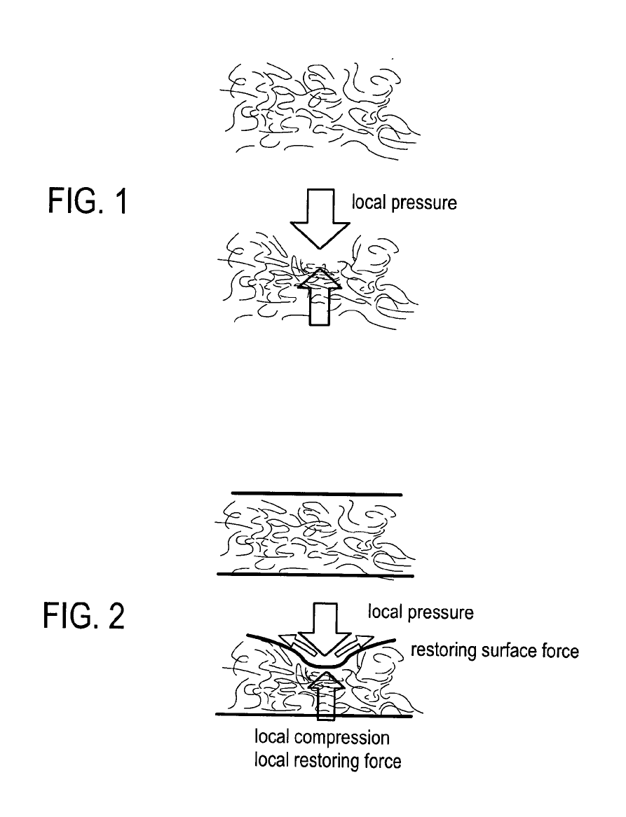

FIG. 1 illustrates the behavior of an Airlaid material without a surface layer comprising interconnected fibers exposed to local pressure.

FIG. 2 illustrates the behavior of an Airlaid material with a surface layer comprising interconnected fibers exposed to local pressure.

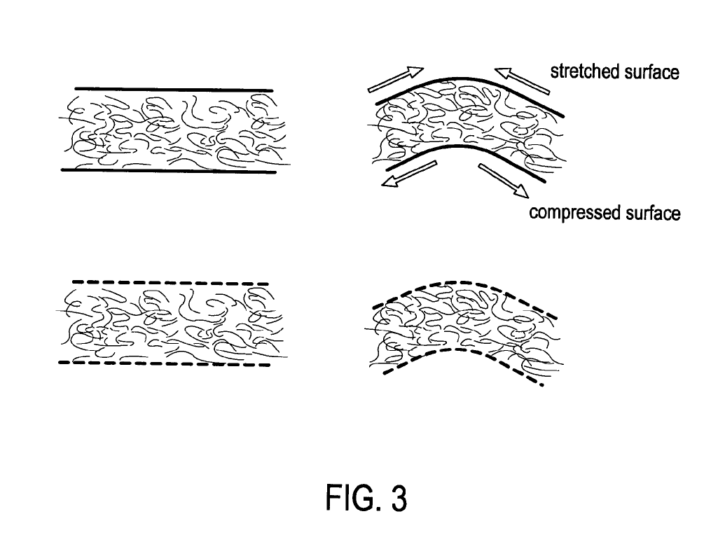

The Airlaid material depicted in FIG. 3 is similar to the material depicted in FIG. 2 with an additional perforation of the surface layers.

FIG. 3 illustrates how the perforation of the surfaces of the Airlaid structure decreases the stiffness of the material. The perforation points form kink or bending points at which the material can be bent without excessively bending the surfaces between these kink or bending points. For a material only perforated in particular zones these zones form areas of enhanced flexibility. This gives the opportunity to create structures with well defined deformation zones which bend into a predetermined direction when a defined force is applied.

This allows the design of hygiene articles with improved wear comfort (diapers, sanitary napkins, incontinence products), wherein the material can adapt to the respective form of the article and can adjust shape in a well defined manner if a force is applied.

Compared to the method of embossing or local stretching (ring-rolling or other mechanical treatment technologies, see patents above) the surface perforation provides the advantage that the Airlaid structure, which is important for liquid transport, is not altered and desired pore structures (e.g. pore size gradient) are not changed.

Manufacturing hygiene products, a local perforation can be integrated into the process e.g. by using needle rollers with a pattern according to the desired perforation structure. Such integration of the perforation step onto the manufacturing line (converter) offers the advantage that the placement of the respective perforated zones is well positioned in the hygiene product. Furthermore, it is avoided that perforated materials with irregular textile properties have to be transported over longer distances on the converting line.

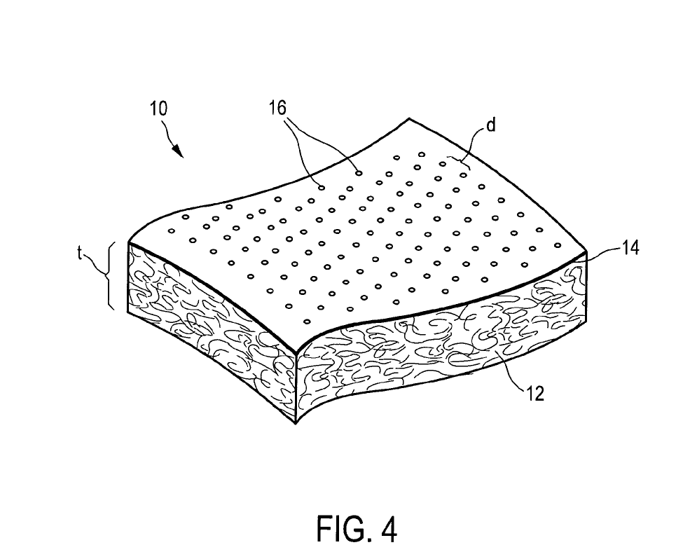

FIG. 4 shows an exemplary embodiment of a fibrous porous fluid absorbent material 10 with a core 12 and a surface layer 14. The core 10 comprises nonwowen fibers. At least 50% of the fibers are cellulosic fibers. The core is produced by an Airlaid process and thus the fibers are only partly interconnected to each other. The core may comprise further components such as superabsorbent polymers, e.g. superabsorbent fibers.

The surface layer 12 comprises bonded fibers so that the fibers form a contiguous layer made of interconnected fibers. The surface layer 12 is perforated by means of perforation holes 16 that created a desired flexural rigidity. The perforation holes can be created by means of a needle roller.

The perforation holes preferably have a diameter in the order of 0.2 mm to 0.5 mm. The distance d between the perforation holes in the surface layer 16 preferably is between 1 mm and 2 mm. The thickness t of the fibrous porous fluid absorbent material 10 preferably is between 1 mm and 2 mm.

Instead of a perforation by means of perforation holes a perforation made of slits can be provided.

Turning now to experiments made with the fibrous porous fluid absorbent material.

### EXAMPLES

Comparison of the stiffness of laminate of Airlaid with wetlaid tissue paper, a demonstration of the influence of different pore size on both surfaces, with or without perforation (see Table 1). Perforation-treated Material of Table 1 is an exemplary partial, i.e. not totally through, but only perforating the wetlaid tissue paper of the laminate with a quadratic pattern having a edge distance of 3 mm, hole size 0.2 mm. Other patterns (rhombic, hexagonic, etc.) are imaginable and also within scope.
By local perforation in zones of the laminate it is possible to establish targeted deformability in specific zones of the whole absorbent article.

We use EDANA Standard Test Method 90.5=WSP90.5 (05) Standard Test Method for Nonwovens Bending Length, which is known to the person skilled in the art of testing nonwoven materials.

We calculate Flexural Rigidity or Bending Stiffness by equation from the Test Method from Bending Length:

TABLE 1

| | | | Stiffness (Flexural | | | |
|---|---|---|---|---|---|---|
| | | Area | rigidity) | | | |
| | | mass | (mN cm) MD | | CD | |
| Airlaid | | (g/m²) | Tissue | Tissue | Tissue | Tissue |
| Material | Nr. | Ø | downside | topside | downside | topside |
| MH250.111 | 1 | 277 | 324 | 325 | 236 | 230 |
| | 2 | 272 | 235 | 228 | 235 | 256 |
| | 3 | 254 | 294 | 314 | 231 | 220 |
| MH250.111 | 1 | 278 | 217 | 198 | 139 | 168 |
| partial perf- | 2 | 265 | 190 | 215 | 150 | 159 |
| treated | | | | | | |
| VH250.117 | 1 | 255 | 345 | 322 | 211 | 202 |
| | 2 | 249 | 257 | 294 | 199 | 190 |
| VH250.117 | 1 | 255 | 137 | 154 | 165 | 170 |
| partial perf- | 2 | 249 | 154 | 197 | 143 | 174 |
| treated | | | | | | |
| VH250.118 | 1 | 262 | 327 | 208 | 215 | 166 |
| | 2 | 259 | 319 | 173 | 212 | 156 |
| VH250.118 | 1 | 262 | 143 | 116 | 111 | 134 |
| partial perf- | 2 | 259 | 134 | 98 | 124 | 115 |
| treated | | | | | | |

### DESCRIPTION OF PREFERRED EMBODIMENTS

The perforation can easily done by certain perforation roller, having penetrating needles giving the material laminate a suitable hole size, e.g. 0.2 mm, and having certain distance, e.g. between 1 mm and 2 mm.

1. A fibrous porous fluid absorbent material comprising nonwoven fibers made by an Airlaid process, at least 50% of said fibers being cellulosic fibers, said fibrous porous fluid absorbent material comprising a non-perforated core wherein said fibers of said core are non-connected or only partially connected and crosslinked with each other and at least one perforated surface layer having a perforation, wherein said fibers of said surface layer are connected to each other and wherein the flexural rigidity of said fibrous porous fluid absorbent material measured according to EDANA Standard Test Method 90.5 is reduced by at least 20%, preferably by at least 30%, more preferably by at least 40%, most preferably by at least 50%, compared to a corresponding non perforated fibrous porous fluid absorbent material comprising a core and a non perforated surface layer.

2. The fibrous porous fluid absorbent material of claim 1, wherein said fibrous porous fluid absorbent material is a laminate having an asymmetric structure by means of lamination of a nonwoven material with an surface layer as carrier material being one component of the laminate, which can be chosen from the group of wetlaid tissue paper, or another nonwoven.

3. The fibrous porous fluid absorbent material of claim 1 wherein said fibrous porous fluid absorbent material is a laminate having an asymmetric structure by means of lamination of a nonwoven material with an surface layer generated by application of a chemical binder without adding additional material by connecting the fibers at the surface to a continuous network.

4. The fibrous porous fluid absorbent material of claim 2, wherein said fibrous porous fluid absorbent material has perforation holes, said perforation holes having a diameter that is smaller than 3 mm, preferably smaller than 1 mm, more preferably smaller than 0.5 mm, most preferably 0.2 mm.

5. The fibrous porous fluid absorbent material of claim 2, wherein said fibrous porous fluid absorbent material has perforation holes, said perforation holes being arranged with a distance between said perforation holes that is less than 30 mm and more than 0.5 mm, preferably smaller than 15 mm, more preferably smaller than 5 mm, most preferably between 1 mm and 2 mm.

6. The fibrous porous fluid absorbent material of claim 2, wherein said fibrous porous fluid absorbent material has a thickness of less than 10 mm and more than 0.5 mm, preferably less than 5 mm, more preferably less than 3 mm, most preferably between 1 mm and 2 mm.

7. The fibrous porous fluid absorbent material according to claim 1, wherein said perforation of said surface layer is produced by means of a perforation roller.

8. The fibrous porous fluid absorbent material according to claim 1, wherein said perforation is achieved by slits.

9. The fibrous porous fluid absorbent material according to claim 1, wherein the perforation is only provided in distinct zones of said fibrous porous fluid absorbent material.
